(19) United States
(12) Patent Application Publication
(10) Pub. No.: US 2026/0305945 A1
(43) Pub. Date: Oct. 8, 2026

(54) MAPPING ELECTRODES FROM MAGNETIC RESONANCE IMAGING (MRI) DATA

(71) Applicant: REGENTS OF THE UNIVERSITY OF MICHIGAN, Ann Arbor, MI (US)

(72) Inventors: David Joseph Brang, Avon Lake, OH (US); Abbey Joy Roelofs, Ypsilanti, MI (US)

(21) Appl. No.: 19/641,921

(22) Filed: Apr. 8, 2026

Related U.S. Application Data
(60) Provisional application No. 63/785,486, filed on Apr. 8, 2025.

(51) Int. Cl. A61B 5/00; A61B 5/055; A61B 5/06; A61B 34/10; A61B 34/20

(52) U.S. Cl. CPC A61B 5/0042; A61B 5/0037; A61B 5/055; A61B 5/062; A61B 5/4064; A61B 5/7267; A61B 5/743; A61B 34/10; A61B 34/20; A61B 2034/105; A61B 2034/2051

(57) ABSTRACT

The following relates generally to methods and system for determining the location of an electrode grid that has been implanted on the brain of a patient in a model of the brain comprising receiving magnetic resonance imaging data, creating a model of a patient's brain, receiving a request to determine a location of an electrode grid, and determining the location of the electrode grid.

### CROSS REFERENCE TO RELATED APPLICATIONS

This application claims the benefit of U.S. Provisional Application No. 63/785,486, entitled “Mapping Electrodes from Magnetic Resonance Imaging (MRI) Data” (filed April 8, 2025), the entirety of which is incorporated by reference herein.

### FIELD

The present disclosure generally relates to methods and systems for mapping electrodes to particular locations on the brain of a patient.

### BACKGROUND

Electrocorticography (ECoG) is an electrophysiological monitoring technique in which electrodes are placed subdurally along the cortical surface of the brain during neurosurgery. ECoG electrodes are used to record electrical signals generated by local populations of neurons offering higher spatial resolution than non-invasive techniques such as electroencephalography (EEG). Historically, ECoG has been used predominantly in patients with epilepsy to localize epileptogenic zones and to electrically stimulate regions of the brain to minimize clinical deficits following tissue resection. Here, electrodes are implanted chronically and ECoG is measured extraoperatively during a one-to-two-week period for the purpose of detecting and localizing the anatomical onset of a seizure, which it enables with high success. In the context of patients with brain tumors, ECoG is clinically used to localize sensory and cognitive processes to aid in the sparing of critical functions, minimizing postoperative deficits.

Traditional methods for ECoG electrode localization were developed for the extraoperative examination of patients with epilepsy. In this situation, electrodes are localized using a postoperative computed tomography (CT) scan that is registered to a patient's preoperative magnetic resonance imaging (MRI) scan. As metallic objects show as bright white artifacts on a CT, this technique enables the accurate identification of individual electrode contacts. Several methods have been developed to facilitate this process and include methods to counteract post-operative brain drift that can occur from chronic implantations. However, CT scans are rarely acquired for patients who receive intraoperative ECoG because the electrodes are not implanted chronically and obtaining a CT would lengthen the duration of the surgery (adding additional risk to the patient) without clinical necessity. As a result, intraoperative ECoG localization requires alternative approaches.

In conventional methods for intraoperative ECoG registration, individual contacts may be localized based on intraoperative photographs taken with and without the grids implanted. In this process, a skilled reviewer (typically the implanting neurosurgeon) will identify the location of individual electrode contacts relative to gyral anatomy which provides a general atlas of labels for electrodes (e.g., contacts 1-3 on the grid were along motor cortex). This process does not allow fine-grained examination of anatomical positions or the projection of anatomical coordinates into a common space. Moreover, ECoG grids can slide under the skull meaning that some contacts are not visible through the craniotomy window. Alternatively, grids can be drawn onto 2D images of template brains providing a rough estimation of their locations, but with unknown accuracy.

A critical step in the analysis of intracranial electroencephalography (iEEG) data is defining the anatomic location of each electrode accurately and efficiently. Accurate electrode localization is critical for neuroscience inference, but efficiency is also important, as a single patient may be implanted with hundreds of electrodes.

Accordingly, there is a pressing need for an ECoG localization technique that does not require a postoperative CT and that is appropriate for patients with altered neuroanatomy such as patients with a brain tumor. The present disclosure addresses the unique challenges of localizing ECoG electrodes in patients with brain tumors, allowing clinicians to accurately align and place electrodes with minimal training, enhancing the accuracy of electrode localization and facilitating more effective and targeted neurosurgical interventions.

### SUMMARY

The present embodiments may be related to mapping the location of electrodes on the brain of a patient for electrodes employed for ECoG during neurosurgery.

In one aspect, a method for mapping electrodes to particular locations on a brain of a patient may comprise receiving magnetic resonance imaging (MRI) data and using that data to create a model of the brain of the patient using the MRI data. A request from a clinician device to determine the location of an electrode grid may be received, followed by determining the location of the electrode grid on the brain using the model. The method may include additional, fewer, or alternate actions, including those discussed elsewhere herein.

In another aspect, a system for mapping electrodes on the brain of a patient may include one or more processors, a MRI machine that comprises at least a gradient coil, a radio frequency (RF) transmit coil, and an RF receive coil. The system may further include one or more memories that include computer executable instructions stored therein that, when executed by the one or more processors, cause the one or more processors to: receive magnetic resonance imaging (MRI) data; create a model of the brain of the patient using the MRI data; receive, from a clinician device, a request to determine a location of an electrode grid; and determine a location of an electrode grid on the brain of the patient using the model. The computer system may include additional, less, or alternate functionality, including that discussed elsewhere herein.

### BRIEF DESCRIPTION OF THE DRAWINGS

Advantages will become more apparent to those skilled in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

The figures described below depict various aspects of the applications, methods, and systems disclosed herein. It should be understood that each figure depicts an embodiment of a particular aspect of the disclosed applications, systems and methods, and that each of the figures is intended to accord with a possible embodiment thereof. Furthermore, wherever possible, the following description refers to the reference numerals included in the following figures, in which features depicted in multiple figures are designated with consistent reference numerals.

FIG. 1 depicts an example computing system for carrying out the described method for determining the placement of an electrode grid on a brain, according to an embodiment.

FIG. 2 depicts an example method for determining the placement of an electrode grid on a brain.

FIGS. 3 and 4 depict example display screens generated during the electrode mapping process.

FIG. 5 depicts an example process flow for applying a machine learning algorithm to determine electrode placement.

### DETAILED DESCRIPTION

The present embodiments relate to, inter alia, systems and methods for determining the placement of an electrode grid on a brain.

### Exemplary Computer System

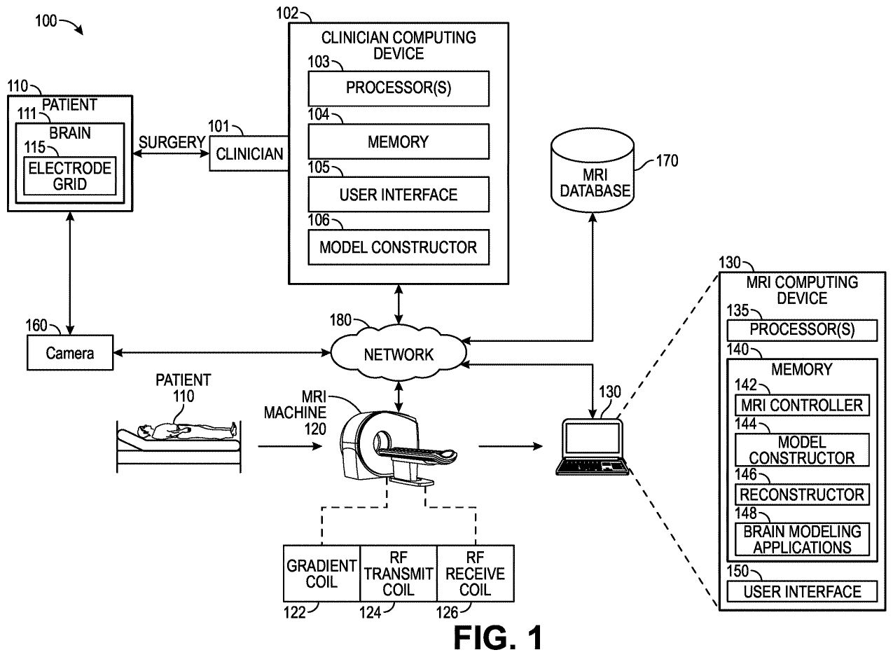

FIG. 1 illustrates an exemplary computer system 100 for mapping the location of electrodes on the brain 111 of a patient, including patient/subject 110 entering a MRI machine 120. The patient 110 includes a brain 111 that will have an electrode grid 115 implanted during surgery. The clinician 101 may operate a clinician computing device 102 that is connected to network 180 to view the MRI data taken with MRI machine 120. The clinician 101 may be a surgeon or other medical professional that provides medical care to a patient. Clinician computing device 102 may include one or more processors 103, such as one or more microprocessors, controllers, and/or any other suitable type of processor. The clinician computing device 102 may further include a memory 104 (e.g., volatile memory, non-volatile memory) accessible by the one or more processors 103 (e.g., via a memory controller). Additionally, the clinician computing device 102 may include a user interface 105, which may be controlled by the one or more processors 103 to display, for example, MRI images of the patient 110. Model constructor 106 may also be included in clinician computing device 102 for constructing models of patient's 110 brain 111 and surrounding tissue, e.g. vasculature and the dura.

Camera 160 may be used to take images of the surgical area on brain 111 both before and after implantation of electrode grid 115. The use of a camera provides visual cues and landmarks for locating electrode grid 115 and comparing to the constructed model of the brain 111. For example, the vasculature may advantageously provide a landmark of a certain shape or size that appears both in the reconstructed model of the brain 111 and digital images of the physical brain 111. The camera 160 may also communicate with the clinician computing device 102 using network 180 so that camera images may be viewed on the same device as MRI data and model constructions.

The MRI machine 120 may produce k-space data that is analyzed and processed by the MRI computing device 130. For example, the MRI machine 120 (and/or the one or more processors 135) may control the gradient coil 122, the RF transmit coil 124 and the RF receive coil 126 to produce the k-space data. The k-space data may then be reconstructed by the reconstructor 146 to form an MRI image. It should be understood that although the example of FIG. 1 illustrates one gradient coil 122, one RF transmit coil 124, and one RF receive coil 126, any number of gradient coils 122, RF transmit coils 124 and/or RF receive coils 126 may be used.

The MRI computing device 130 may include one or more processors 135 such as one or more microprocessors, controllers, and/or any other suitable type of processor. The MRI computing device 130 may further include a memory 140 (e.g., volatile memory, non-volatile memory) accessible by the one or more processors 135 (e.g., via a memory controller). Additionally, the MRI computing device 130 may include a user interface 150, which may be controlled by the one or more processors 135 to display, for example, MRI images of the patient 110. The user interface 150 may further be used to control (e.g., via the MRI controller 142) the MRI machine 120 (e.g., control the gradient coil 122, the RF transmit coil 124, the RF receive coil 126, etc. by administering a pulse sequence and/or receiving signals).

The MRI data may also be obtained from more than one MRI machines or using more than one MRI imaging technique, such as T1- and T2-weighted MRI scans and/or post gadolinium contrast MRI. Post gadolinium MRI may be especially useful for obtaining information related to the vasculature of the patient's 110 brain. Vasculature imaging may be used to provide anatomical landmarks, which may advantageously improve electrode placement. Using a plurality of MRI techniques may provide a more complete reconstruction of the patient's 110 brain 111 to aid in electrode location.

The one or more processors 135 may interact with the memory 140 to obtain, for example, computer-readable instructions stored in the memory 140. Additionally or alternatively, computer-readable instructions may be stored on one or more removable media (e.g., a compact disc, a digital versatile disc, removable flash memory, etc.) that may be coupled to the MRI computing device 130 to provide access to the computer-readable instructions stored thereon. In particular, the computer-readable instructions stored on the memory 140 may include instructions for executing various applications, such as a, MRI controller 142, MRI model constructor 144, reconstructor 146, and/or brain modeling applications 148. Brain modeling applications 148 may include conventional software such as Freesurfer, Advanced Normalization Tools, or other modeling tools.

Furthermore, the MRI computing device 130 may be connected to the MRI machine 120 and/or MRI database 170 via network 180, which may be a wired or wireless network, such as the internet. The MRI database 170 may store any MRI data, such as fully or partially sampled reference MRI data.

### Exemplary Computer-Implemented Method for Mapping Electrodes on a Patient's Brain

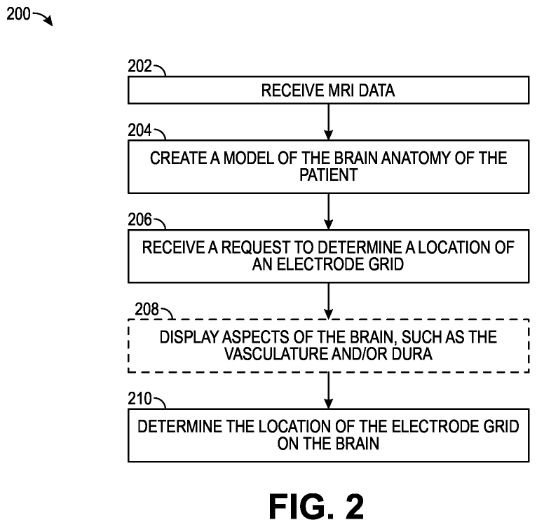

FIG. 2 depicts an example process flow for mapping the location of electrodes on the brain 111 of a patient 110. At block 202, the MRI computing device 130 receives MRI data of the brain of patient 110 from MRI machine 120 collected using gradient coil 122, RF transmit coil 124, and RF receive coil 126. At block 204, the model constructor 144 creates a model of the brain 111 of patient 110. The model may include a cortical reconstruction of one or both hemispheres of the brain of patient 110, along with the dura, vasculature, or other physical structures. Additionally, the model may be a two-dimensional (2D) surface cortical reconstruction or a brain-extracted three-dimensional (3D) volumetric model. A reconstruction of the vasculature is optional, but may be very helpful for providing anatomical landmarks to aid placing the electrode grid 115 location in the software.

The modeling of brain 111 may be accomplished using pre-loaded models of a brain that are based on average brain features, such as the MNI Coordinate System template or Talairach coordinates, but it is preferred to use individual patient anatomy. The MNI or other template may be used if the patient's brain 111 is not available, which may be due to large anatomical defects or unusable MRI scans. The MRI data of the brain of patient 110 may be converted to a model using conventional software programs for this purpose, such as Advanced Normalization Tools Brain Extractor or Freesurfer. The vasculature of the brain may be obtained using Post-Gadolinium MRI imaging or other suitable techniques.

The dura may be reconstructed as part of the brain model to prevent the software from determining that the electrodes have ‘fallen’ into the sulci of the brain. By doing this, the electrodes will sit on top of the dura when they are added to the model. Using a model of the dura helps to ensure that the modeled and actual location of the electrodes match in the x, y, and z directions. It should also be understood that the brain 111 may not be the same shape during surgery as it was during the preoperative MRI scan. This may happen because the brain 111 may deform or protrude out of the skull if there is intercranial pressure during surgery when the brain 111 is exposed.

At block 206, a request to determine the location of an electrode grid 115 is received. For example, the clinician 101 may enter the request via the user interface 105, which may be received by one or both of the clinician computing device 102 and/or the MRI computing device 130.

At optional block 208, different aspects of the brain 111 modeling may be displayed, such as the vasculature or the dura. As described above, the vasculature may provide anatomical landmarks to aid in electrode grid 115 placement. The dura may not be required to be displayed beyond an initial check that it has been correctly added to the model.

At block 210, the location of the electrode grid 115 on the brain model is determined by manually adjusting the grid 115 location in the software. This process can be aided by comparing the brain 111 model, including the vasculature, gyri, and sulci, with images taken with camera 160 intraoperatively both with and without the electrode grid placed on the brain 111 of patient 110. Using the anatomical landmarks may improve accuracy of the electrode placement and thus better management of neurological symptoms and higher rates of surgical success. The method 200 may further be used to place more than one electrode grid 115 on the brain 111 model.

In an alternative to locating the electrode grid 115 manually, neuronavigation coordinates may be used. Neuronavigation uses the pre-operative or intra-operative brain imaging to create a 3D model of the brain 111. The 3D model is registered with the patient 110 anatomy during surgery using an intraoperative tracker, which may be used to create a coordinate system. The coordinates may then be used for locating the electrode grid 115. This method may be used for precise electrode grid 115 placement, but may not be available in all cases.

In another embodiment, an artificial intelligence (AI) or machine learning (ML) training application may be used for initial placement of the electrode grid 115, which may then be manually adjusted and corrected. The ML application may be stored in memory 104 of clinician computing device 102 or memory 140 of MRI computing device 130. For example, as will be described elsewhere herein, an AI or ML training application may receive historical data related to brain anatomy, tumor identification, and electrode placement, into the AI or ML algorithm to train the electrode placement ML algorithm.

### Exemplary Displays

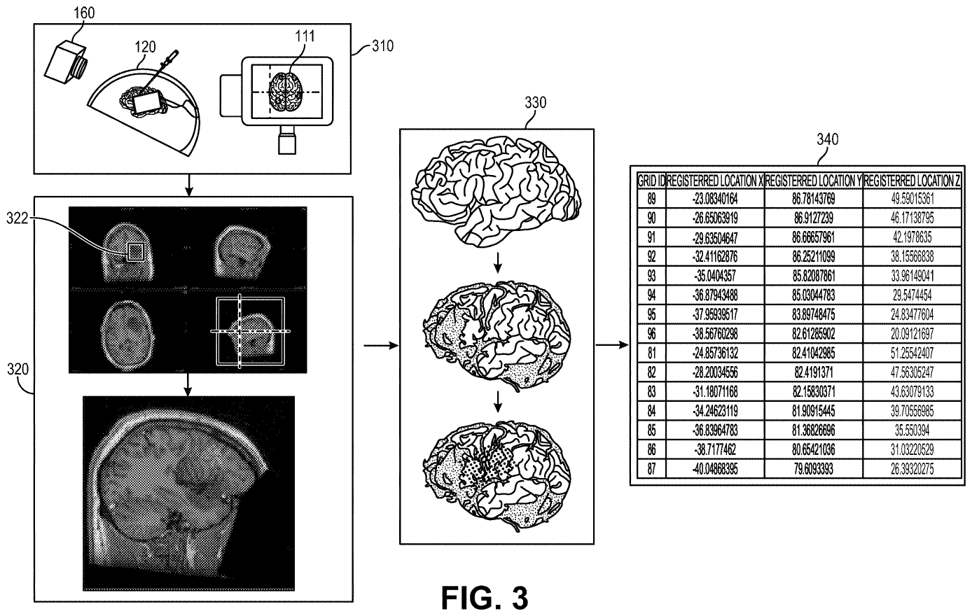

FIG. 3 depicts an example implementation 300 of method 200. At block 310, imaging is obtained using camera 160 and MRI machine 120 of brain 111. In block 310, brain 111 is shown with the electrode grid 115 already implanted, but camera 160 images may be acquired both before and after grid 115 implantation. In some embodiments, neuronavigational tools may be used to localize the corners of the grid 115 in real time. The use of these tools allows the clinician 101 to locate the corners of the electrode grid 115 when it is placed to aid in locating it in the software later. In some cases, the corners of the electrode grid 115 are not visible because they may slide under the skull and so are not able to be localized in real time by the clinician 101.

Block 320 depicts a model of the brain 111 using the MRI data. The modeling of the brain 111 may be performed using MRI viewing software. At this block, the tumor or lesion 322 may be localized and the vasculature may be viewed. The lower image shows a part of brain image obtained with camera 160 overlaid with the brain model.

Block 330 depicts an example brain model. The top image shows the brain model, including the sulci and gyri of the imaged brain 111. The middle image shows the vasculature overlaid on the model of brain 111. The bottom image shows the placement of the electrode grid on the brain 111 that corresponds to the location of the electrode grid 115 on the patient 110.

Block 340 depicts example coordinates that may be output from the computer system that corresponds to the location of the electrodes that form the electrode grid 115 that was implanted on the patient 110. The electrode location may then be used for further analysis.

Once the electrode grid 115 is placed at certain location, it may be used for other purposes during surgery, such as to determine further treatment, e.g. administering stimulation to a particular location of the brain. The electrode grid may also be used for testing for certain brain functions, e.g. speech, motor, vocational functioning to give real-time feedback to the clinician about neural activity. Real-time analysis as facilitated by accurate electrode placement allows for projecting neural activity back onto the brain during surgery through testing, which then allows a clinician to more precisely map out what areas of the brain are responsive to certain tasks and in turn then helps the clinician accurately determine how much brain tissue needs to be removed. By enabling faster and more precise electrode placement, the present methods and systems enable a clinician to remove less brain matter and preserve more healthy brain tissue. This may be especially advantageous for brain tumors in insular regions of the brain and allow for the least removal of healthy brain regions to preserve brain function.

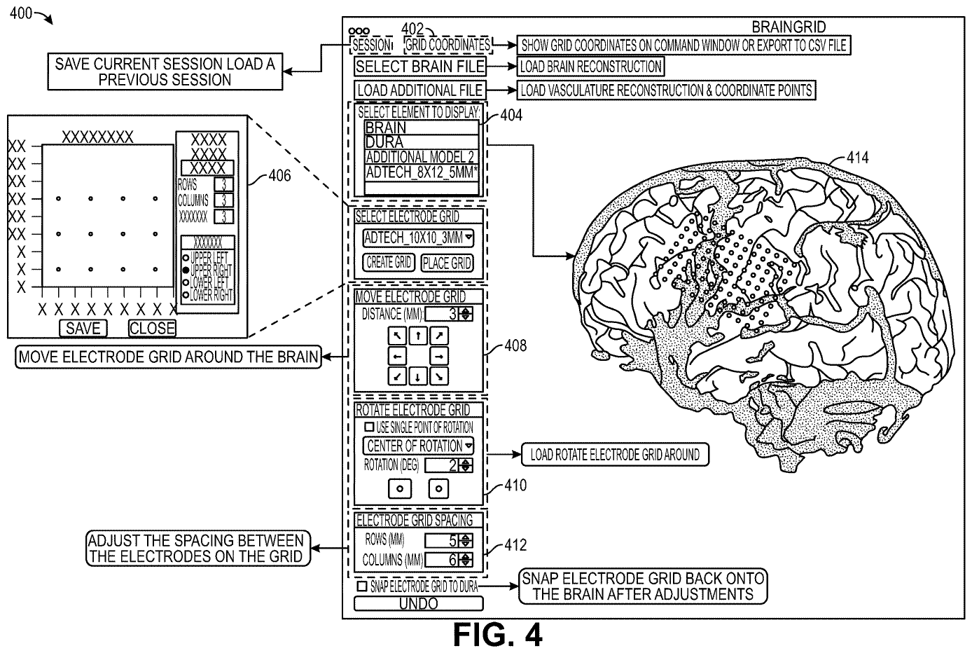

FIG. 4 depicts an exemplary model of brain 111 including available options for aiding in the location of electrode grid 115 on the brain 111 of a patient 110. On the right of the figure, a model of a brain 111 is shown that includes the vasculature and the location of the electrode grid 115, similar to that shown in FIG. 3. Option 402 relates to grid 115 coordinates and allows for grid coordinates to be shown on the main, or command, window. The grid coordinates may also be exported in a standard format, such as csv. Option 402 also includes buttons for selecting a brain file that loads a brain model and an option to load an additional file that may be used to load a vasculature model and coordinate points related to the vasculature.

Option 404 allows the software user (e.g., clinician 101) to select which elements to display, such as the brain model, dura, vasculature, or additional brain models. Option 406 relates to the selecting an electrode grid 115 for placing in the software. When selected, a pop-up window may open that shows the various options for electrode grids, such as the size of the grid, number of electrodes, horizontal and vertical spacing between electrodes, and the number of rows and columns of electrodes. The user may also be able to choose which electrode of the grid is labeled as electrode 1 for counting purposes. Because a grid may not be regularly shaped, it may be necessary to remove an electrode in the software to ensure that the modeled grid and actual implanted grid 115 have the same number of electrodes.

Initial placement of the electrode grid 115 may be done using neural coordinates obtained during surgery or by manual selection of a point on the model. The neural coordinates may be inputted as XYZ coordinate values, which may be more accurate than manual placement. Manual placement may be performed using visual anatomic or vascular correspondence to intraoperative photos.

The initial placement of the electrode grid 115 may not always be exact, and some adjustment may be required. Option 408 allows the user to move the electrode grid 115 around the reconstructed brain 111 using arrow keys. The user may also select the distance that the grid 115 moves with each click of an arrow button, allowing for highly accurate placement of the electrode grid 115. Option 410 allows for rotation of the electrode grid 115, including allowing the user to specify the direction of rotation and/or number of degrees to rotate. Users may also select if the electrode grid is rotated around a single point of rotation or the center of the grid. Using a single point of rotation may be useful when one point on the electrode grid 115 has been placed using anatomical landmarks and should not be moved.

Option 412 relates to adjusting the electrode grid spacing, similar to the menu in option 406. The user can selection the spacing between rows and columns of electrodes, which corresponds to horizontal and vertical spacing between electrodes or rows. Below that option is the selection for snapping the electrode grid 115 back on the brain 111 after adjustments are made by the user. This prevents the software from determining that the electrode contacts have fallen into the tissue.

Other options, which are not shown, relating to electrodes include the type of electrode, diameter of electrodes, and shape of the electrodes. The shape and diameter may not be consistent for the entire electrode grid, e.g., there may be a clinical advantage for some electrodes to be sized differently from other electrodes in the grid.

Element 414 depicts a model of brain 111 that has the vasculature displayed and that an electrode grid 115 has been placed. The display of element 414 may include other or fewer elements depending on the options selected by the user, such as toggling the display of the brain vasculature or the dura. In some embodiments, an option, not shown, may be integrated into the software for rotating the brain model to aid in visualizing the brain and correctly placing the electrode grid 115. Viewing the brain model from different angles may be advantageous for correctly placing the electrode grid 115.

Other possible actions include a session button for saving the current session or loading a previous session. If multiple grids have been implanted and need to be located, the user may add another electrode grid using the same process as for a single electrode grid 115. Each grid may be given a unique label and/or coloring for ease of distinguishing between them.

It should be understood that not all blocks and/or events of the exemplary diagrams and/or flowcharts are required to be performed. Moreover, the exemplary diagrams and/or flowcharts are not mutually exclusive (e.g., block(s)/events from each example diagram and/or flowchart may be performed in any other diagram and/or flowchart). The exemplary diagrams and/or flowcharts may include additional, less, or alternate functionality, including that discussed elsewhere herein.

### Exemplary AI and/or ML Techniques

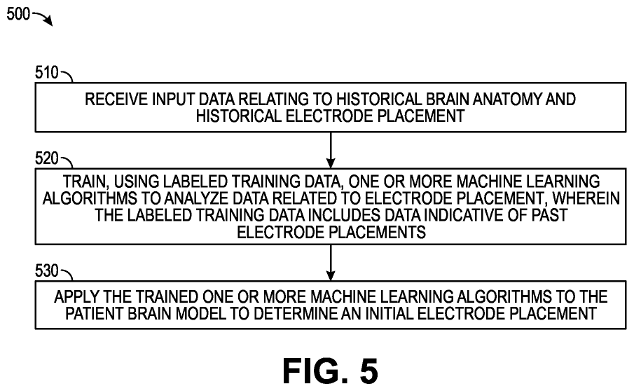

FIG. 5 depicts an example flowchart for training and deploying an AI/ML model for determining an initial electrode grid 115 placement. Although the following discussion refers to ML models, it should be understood that the model may additionally or alternatively be an AI model (e.g., the following discussion applies equally to an AI and/or ML model).

At block 510, training data may be received at the one or more processors 103. The training data may be related to historical electrode placements and brain anatomy. The historical brain anatomy may include brains that have tumors or lesions present and brains that do not. The example historical brain anatomy may include MRI or other suitable data related to brain structure or shape. Historical electrode placement training data may include the location of electrode grids that have been implanted on patients'brains in the past with relation to identified tumors or lesions or other anatomical data, such as vasculature, the dura, or cranial openings. This list is for example only, it should be understood that there may be other historical anatomical and electrode grid placement data that may be used as training data for a ML algorithm related to initial electrode grid placements.

In some examples, the training data comprises: (i) inputs to the machine learning model (e.g., also referred to as independent variables, or explanatory variables), and/or (ii) outputs of the machine learning model (e.g., also referred to as dependent variables, or response variables). In some such examples, the dependent variables are the historical electrode grid placements that the ML algorithm is trained to determine; and the independent variables (e.g., historical brain anatomical data) are used to determine the dependent variables (e.g., historical initial electrode grid placements). Put another way, the independent variables may have an impact on the dependent variables; and the ML algorithms may be trained to find this impact.

At block 520, an ML algorithm is trained using the training data related to (1) historical brain anatomical structures, historical brain models, and historical electrode grids (e.g., the independent variables); and (2) historical electrode grid placements (e.g., the dependent variables). The historical electrode grids may include specifying the number and spacing of electrodes in the historical grids. In some examples, the training data may be labeled as examples of correct and incorrect electrode grid placements. The training data may also be labeled as examples of correct and incorrect tumor identification. For example, because the presence of a tumor or lesion may cause signal dropout, dark spots in the reconstruction may be classified as possible tumors or lesions.

At block 530, the trained ML algorithm may be applied to the brain 111 of patient 110 data received from MRI machine 120. The trained ML algorithm may then output initial electrode grid 115 placements for the clinician 101 to view and correct as needed.

It should be noted that the described ML training techniques may be applied to other aspects of the present invention, such as improved anatomical extraction and identification from the MRI data. This may include vasculature, tumors or lesions, or other anatomical structures.

It should be understood that not all blocks and/or events of the exemplary signal diagrams and/or flowcharts are required to be performed. Moreover, the exemplary signal diagrams and/or flowcharts are not mutually exclusive (e.g., block(s)/events from each example signal diagram and/or flowchart may be performed in any other signal diagram and/or flowchart). The exemplary signal diagrams and/or flowcharts may include additional, less, or alternate functionality, including that discussed elsewhere herein.

The described method and system address a critical gap in the field of neurosurgery and neuroscience research by providing a reliable method for localizing ECoG electrodes in patients with brain tumors. Traditional localization techniques, adequate for epilepsy but insufficient for tumor-involved cases, are effectively supplemented by the described methods and system use of intraoperative photography and vascular mapping. The described methods and system may be used to enhance the surgical interventions and increase understanding of the interplay between brain function and disease.

### Additional Exemplary Embodiments

Aspect 1. A computer-implemented method for mapping electrodes to particular locations on a brain of a patient, comprising:
receiving, via one or more processors, magnetic resonance imaging (MRI) data;
creating, via the one or more processors, a model of the brain of the patient using the MRI data;
receiving, via the one or more processors, from a user device, a request to determine a location of an electrode grid; and
determining, via the one or more processors, the location of the electrode grid on the brain using the model.

Aspect 2. The computer-implemented method of aspect 1, wherein the MRI data includes vasculature data.

Aspect 3. The computer-implemented method of any one of aspects 1-2, wherein the MRI data is collected using post-gadolinium contrast MRI image.

Aspect 4. The computer-implemented method of any one of aspects 1-3, wherein the model of the brain includes a dura and a brain vasculature.

Aspect 5. The computer-implemented method of any one of aspects 1-4, wherein the brain model is a 2D surface cortical reconstruction or a brain-extracted 3D volumetric model.

Aspect 6. The computer-implemented method of aspect 1, wherein the location of more than one electrode grid is determined.

Aspect 7. The computer-implemented method of any one of aspects 1-6, wherein the received request includes: (i) a number of electrodes in the electrode grid, (ii) a horizontal spacing between electrodes in the electrode grid, and/or (iii) a vertical spacing between the electrodes in the electrode grid.

Aspect 8. The computer-implemented method of any one of aspects 1-7, further comprising:
training, by the one or more processors, one or more machine learning algorithms by inputting, into the one or more machine learning algorithms, labeled training data including data indicative of historical: (i) brain anatomical structures and (ii) electrode grid placements; and
wherein determining the location of the electrode grid on the brain using the model further includes applying the trained one or more machine learning algorithms to the model of the brain data.

Aspect 9. A system for mapping electrodes to particular locations on a brain of a patient, comprising:
one or more processors;
a magnetic resonance imaging (MRI) machine, the MRI machine comprising:
   a gradient coil;
   at least one radio frequency (RF) transmit coil; and
   at least one RF receive coil; and
one or more memories coupled to the one or more processors;
the one or more memories including computer executable instructions stored therein that, when executed by the one or more processors, cause the one or more processors to:
   receive magnetic resonance imaging (MRI) data;
   create a model of the brain of the patient using the MRI data;
   receive, from a user device, a request to determine a location of an electrode grid; and
   determine a location of an electrode grid on the brain of the patient using the model.

Aspect 10. The system of aspect 9, wherein the MRI data includes vasculature data.

Aspect 11. The system of any one of aspects 9-10, wherein the model of the brain includes a dura and a brain vasculature.

Aspect 12. The system of any one of aspects 9-11, wherein the brain model is a 2D surface cortical reconstruction or a brain-extracted 3D volumetric model.

Aspect 13. The system of any one of aspects 9-12, wherein the location of more than one electrode grid is determined.

Aspect 14. The system of any one of aspects 9-13, wherein the received request includes: (i) a number of electrodes in the electrode grid, (ii) a horizontal spacing between electrodes in the electrode grid, and/or (iii) a vertical spacing between the electrodes in the electrode grid.

Aspect 15. The system of any one of aspects 9-14, wherein the computer executable instructions, when executed by the one or more processors, further cause the one or more processors to:
train, by the one or more processors, one or more machine learning algorithms by inputting, into the one or more machine learning algorithms, labeled training data including data indicative of historical: (i) brain anatomical structures and (ii) electrode grid placements; and
wherein determining the location of the electrode grid on the brain using the model further includes applying the trained one or more machine learning algorithms to the model of the brain data.

Aspect 16. The system of any one of aspects 9-15, wherein the MRI data is collected using post-gadolinium contrast MRI image.

Aspect 17. A tangible, non-transitory computer-readable medium storing instructions for mapping electrodes to particular locations on a brain of a patient, when executed by one or more processors of a computing device, cause the computing device to:
receive magnetic resonance imaging (MRI) data;
create a model of the brain of the patient using the MRI data;
receive, from a user device, a request to determine a location of an electrode grid; and
determine a location of an electrode grid on the brain of the patient using the model.

Aspect 18. The non-transitory computer-readable medium of aspect 17, wherein the model of the brain includes a dura and a brain vasculature.

Aspect 19. The non-transitory computer-readable medium of any one of aspects 17-18, wherein the location of more than one electrode grid is determined.

Aspect 20. The non-transitory computer-readable medium of any one of aspects 17-19, wherein the received request includes: (i) a number of electrodes in the electrode grid, (ii) a horizontal spacing between electrodes in the electrode grid, and/or (iii) a vertical spacing between the electrodes in the electrode grid.

### Other Matters

Although the text herein sets forth a detailed description of numerous different embodiments, it should be understood that the legal scope of the invention is defined by the words of the claims set forth at the end of this patent. The detailed description is to be construed as exemplary only and does not describe every possible embodiment, as describing every possible embodiment would be impractical, if not impossible. One could implement numerous alternate embodiments, using either current technology or technology developed after the filing date of this patent, which would still fall within the scope of the claims.

It should also be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based upon any statement made in any section of this patent (other than the language of the claims). To the extent that any term recited in the claims at the end of this disclosure is referred to in this disclosure in a manner consistent with a single meaning, that is done for sake of clarity only so as to not confuse the reader, and it is not intended that such claim term be limited, by implication or otherwise, to that single meaning.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (code embodied on a non-transitory, tangible machine-readable medium) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC) to perform certain operations). A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment may be included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

Upon reading this disclosure, those of skill in the art will appreciate still additional alternative structural and functional designs for the approaches described herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

The particular features, structures, or characteristics of any specific embodiment may be combined in any suitable manner and in any suitable combination with one or more other embodiments, including the use of selected features without corresponding use of other features. In addition, many modifications may be made to adapt a particular application, situation or material to the essential scope and spirit of the present invention. It is to be understood that other variations and modifications of the embodiments of the present invention described and illustrated herein are possible in light of the teachings herein and are to be considered part of the spirit and scope of the present invention.

While the preferred embodiments of the invention have been described, it should be understood that the invention is not so limited and modifications may be made without departing from the invention. The scope of the invention is defined by the appended claims, and all devices that come within the meaning of the claims, either literally or by equivalence, are intended to be embraced therein.

It is therefore intended that the foregoing detailed description be regarded as illustrative rather than limiting, and that it be understood that it is the following claims, including all equivalents, that are intended to define the spirit and scope of this invention.

Furthermore, the patent claims at the end of this patent application are not intended to be construed under 35 U.S.C. § 112(f) unless traditional means-plus-function language is expressly recited, such as “means for” or “step for” language being explicitly recited in the claim(s). The systems and methods described herein are directed to an improvement to computer functionality, and improve the functioning of conventional computers.

What is claimed:

1. A computer-implemented method for mapping electrodes to particular locations on a brain of a patient, comprising:
receiving, via one or more processors, magnetic resonance imaging (MRI) data;
creating, via the one or more processors, a model of the brain of the patient using the MRI data;
receiving, via the one or more processors, from a user device, a request to determine a location of an electrode grid; and
determining, via the one or more processors, the location of the electrode grid on the brain using the model.

2. The computer-implemented method of claim 1, wherein the MRI data includes vasculature data.

3. The computer-implemented method of claim 2, wherein the MRI data is collected using post-gadolinium contrast MRI image.

4. The computer-implemented method of claim 1, wherein the model of the brain includes a dura and a brain vasculature.

5. The computer-implemented method of claim 1, wherein the brain model is a 2D surface cortical reconstruction or a brain-extracted 3D volumetric model.

6. The computer-implemented method of claim 1, wherein the location of more than one electrode grid is determined.

7. The computer-implemented method of claim 1, wherein the received request includes: (i) a number of electrodes in the electrode grid, (ii) a horizontal spacing between electrodes in the electrode grid, and/or (iii) a vertical spacing between the electrodes in the electrode grid.

8. The computer-implemented method of claim 1, further comprising:
training, by the one or more processors, one or more machine learning algorithms by inputting, into the one or more machine learning algorithms, labeled training data including data indicative of historical: (i) brain anatomical structures and (ii) electrode grid placements; and
wherein determining the location of the electrode grid on the brain using the model further includes applying the trained one or more machine learning algorithms to the model of the brain data.

9. A system for mapping electrodes to particular locations on a brain of a patient, comprising:
one or more processors;
a magnetic resonance imaging (MRI) machine, the MRI machine comprising:
a gradient coil;
at least one radio frequency (RF) transmit coil; and
at least one RF receive coil; and
one or more memories coupled to the one or more processors;
the one or more memories including computer executable instructions stored therein that, when executed by the one or more processors, cause the one or more processors to:
receive magnetic resonance imaging (MRI) data;
create a model of the brain of the patient using the MRI data;
receive, from a user device, a request to determine a location of an electrode grid; and
determine a location of an electrode grid on the brain of the patient using the model.

10. The system of claim 9, wherein the MRI data includes vasculature data.

11. The system of claim 9, wherein the model of the brain includes a dura and a brain vasculature.

12. The system of claim 9, wherein the brain model is a 2D surface cortical reconstruction or a brain-extracted 3D volumetric model.

13. The system of claim 9, wherein the location of more than one electrode grid is determined.

14. The system of claim 9, wherein the received request includes: (i) a number of electrodes in the electrode grid, (ii) a horizontal spacing between electrodes in the electrode grid, and/or (iii) a vertical spacing between the electrodes in the electrode grid.

15. The system of claim 9, wherein the computer executable instructions, when executed by the one or more processors, further cause the one or more processors to:
train, by the one or more processors, one or more machine learning algorithms by inputting, into the one or more machine learning algorithms, labeled training data including data indicative of historical: (i) brain anatomical structures and (ii) electrode grid placements; and
wherein determining the location of the electrode grid on the brain using the model further includes applying the trained one or more machine learning algorithms to the model of the brain data.

16. The system of claim 9, wherein the MRI data is collected using post-gadolinium contrast MRI image.

17. A tangible, non-transitory computer-readable medium storing instructions for mapping electrodes to particular locations on a brain of a patient, when executed by one or more processors of a computing device, cause the computing device to:
receive magnetic resonance imaging (MRI) data;
create a model of the brain of the patient using the MRI data;
receive, from a user device, a request to determine a location of an electrode grid; and
determine a location of an electrode grid on the brain of the patient using the model.

18. The non-transitory computer-readable medium of claim 17, wherein the model of the brain includes a dura and a brain vasculature.

19. The non-transitory computer-readable medium of claim 17, wherein the location of more than one electrode grid is determined.

20. The non-transitory computer-readable medium of claim 17, wherein the received request includes: (i) a number of electrodes in the electrode grid, (ii) a horizontal spacing between electrodes in the electrode grid, and/or (iii) a vertical spacing between the electrodes in the electrode grid.
